(19) United States
(12) Patent Application Publication
(10) Pub. No.: US 2021/0407201 A1
(43) Pub. Date: Dec. 30, 2021

(54) TIME-OF-FLIGHT (TOF) CAMERA SYSTEMS AND METHODS FOR AUTOMATED DERMATOLOGICAL CRYOSPRAY TREATMENTS

(71) Applicant: R2 Technologies, Inc., San Ramon, CA (US)

(72) Inventors: Rico Stenson, San Mateo, CA (US); Erik Stauber, Albany, CA (US)

(21) Appl. No.: 17/349,756

(22) Filed: Jun. 16, 2021

Related U.S. Application Data
(60) Provisional application No. 63/043,689, filed on Jun. 24, 2020.

(51) Int. Cl. G06T 17/20; A61B 18/02

(52) U.S. Cl. CPC G06T 17/205; A61B 2018/00452; A61B 18/0218

(57) ABSTRACT

Time-of flight camera system and methods for automated dermatological cryospray treatments are disclosed herein. A method of controlling a skin cooling treatment system including a mechanical arm with a cryospray applicator coupled to a distal end of the mechanical arm, can include receiving a point cloud generated from a portion of skin of a patient for receiving a skin cooling treatment and generating a polygon mesh surface representative of the portion of skin of the patient from the point cloud. The polygon mesh surface can include a plurality of linked vertices. The method can include generating waypoints and delivery vectors based on the polygon mesh surface, linking the waypoints to form a treatment path, and delivering a skin treatment to the portion of skin according to the treatment path.

### CROSS-REFERENCES TO RELATED APPLICATIONS

This application claims the benefit of U.S. Provisional Application No. 63/043,689, entitled “TOF CAMERA SYSTEMS AND METHODS FOR AUTOMATED DERMATOLOGICAL CRYOSPRAY TREATMENTS”, and filed Jun. 24, 2020, the entirety of which is incorporated by reference herein.

### BACKGROUND

Cryotherapy is the local or general use of cold in medical therapy. Cryotherapy can include the controlled freezing of biological tissue, which controlled freezing of biological tissue, such as skin tissue, can produce various effects. Certain tissue freezing procedures and devices, such as conventional cryoprobes, can cause severe freezing of tissue and generate cellular and visible skin damage.

There is a demand for cosmetic products that can change the appearance of skin or otherwise controllably affect skin pigmentation. This can include lightening or darkening of the skin. For example, it may be desirable to lighten the overall complexion or color of a region of skin to alter the general appearance for cosmetic reasons. Also, lightening of particular hyperpigmented regions of skin, such as freckles, ‘café au lait’ spots, melasma, or dark circles under the eyes that may result from excessive local amounts of pigment in the skin, may also be desirable for cosmetic reasons. Hyperpigmentation can result from a variety of factors such as UV exposure, aging, stress, trauma, inflammation, etc. Such factors can lead to an excess production of melanin, or melanogenesis, in the skin by melanocytes, which can lead to formation of hyperpigmented areas. Such hyperpigmented areas are typically associated with excess melanin within the epidermis and/or dermal-epidermis junction. However, hyperpigmentation can also result from excess melanin deposited within the dermis.

Hypopigmentation of skin tissue has been observed as a side effect in response to temporary cooling or freezing of the tissue, such as may occur during conventional cryosurgery procedures. Loss of pigmentation following skin cooling or freezing may result from decreased melanin production, decreased melanosome production, destruction of melanocytes, or inhibited transfer or regulation of melanosome into the keratinocytes in the lower region of the epidermal layer. The resultant hypopigmentation may be long-lasting or permanent. However, it has also been observed that some of these freezing procedures can generate regions of hyperpigmentation (or skin darkening) of skin tissue. The level of increase or decrease in pigmentation may be dependent upon certain aspects of the cooling or freezing conditions, including the temperature of the cooling treatment, and the length of time the tissue is maintained in a frozen state.

Improved hypopigmentation treatments, devices, and systems have been developed to improve the consistency of skin freezing and the overall hypopigmentation consistency. For example, it has been observed that moderate degrees of freezing (e.g., −4 to −30 degrees Celsius) at shorter time frames (e.g., 30 to 60 seconds) can produce particular dermatological effects, such as affecting the expression of skin pigmentation (e.g., hypopigmentation). Cryotherapy can be provided using a variety of techniques including the direct application of a cryogen spray to the skin of the patient or the application of a cooled probe or plate to the skin of the patient. Exemplary methods and devices are described in: U.S. Patent Publication No. 2011/0313411, filed on Aug. 7, 2009, and entitled “METHOD AND APPARATUS FOR DERMATOLOGICAL HYPOPIGMENTATION”; U.S. Patent Publication No. 2014/0303696, filed on Nov. 16, 2012, and entitled “METHOD AND APPARATUS FOR CRYOGENIC TREATMENT OF SKIN TISSUE”; U.S. Patent Publication No. 2014/0303697, filed on Nov. 16, 2012, and entitled “METHOD AND APPARATUS FOR CRYOGENIC TREATMENT OF SKIN TISSUE”; U.S. Patent Publication No. 2015/0223975, filed on Feb. 12, 2015, and entitled “METHOD AND APPARATUS FOR AFFECTING PIGMENTATION OF TISSUE”; U.S. Patent Publication No. 2017/0065323, filed on Sep. 6, 2016, and entitled “MEDICAL SYSTEMS, METHODS, AND DEVICES FOR HYPOPIGMENTATION COOLING TREATMENTS”, the entirety of each of which is hereby incorporated by reference herein.

While the treatment of skin or a localized lesion to affect pigmentation can be accomplished with cryotherapy, it may be desirable to provide improved methods, systems, and devices for cryotherapy. In particular, improved designs, controls and parameters associated with cryogen delivery to achieve consistent and reliable skin freezing and desired skin treatment effect may be of benefit. Accordingly, improved dermatological cryospray methods, systems, and devices are desirable.

### BRIEF DESCRIPTION OF THE DRAWINGS

FIG. 1 is a schematic illustration of one embodiment of a skin cooling treatment system.

FIG. 2 is a perspective view of one embodiment of the skin cooling treatment system.

FIG. 3 is a perspective view of one embodiment of the cryospray applicator.

FIG. 4 is a perspective view of another embodiment of the cryospray applicator.

FIG. 5 is an illustration of a point cloud representing a face.

FIG. 6 is an illustration of a plurality of point clouds representing a face.

FIG. 7 is a schematic illustration of one embodiment of a method of multi-perspective point cloud generation.

FIG. 8 is a representation of unmerged, multi-perspective point cloud data.

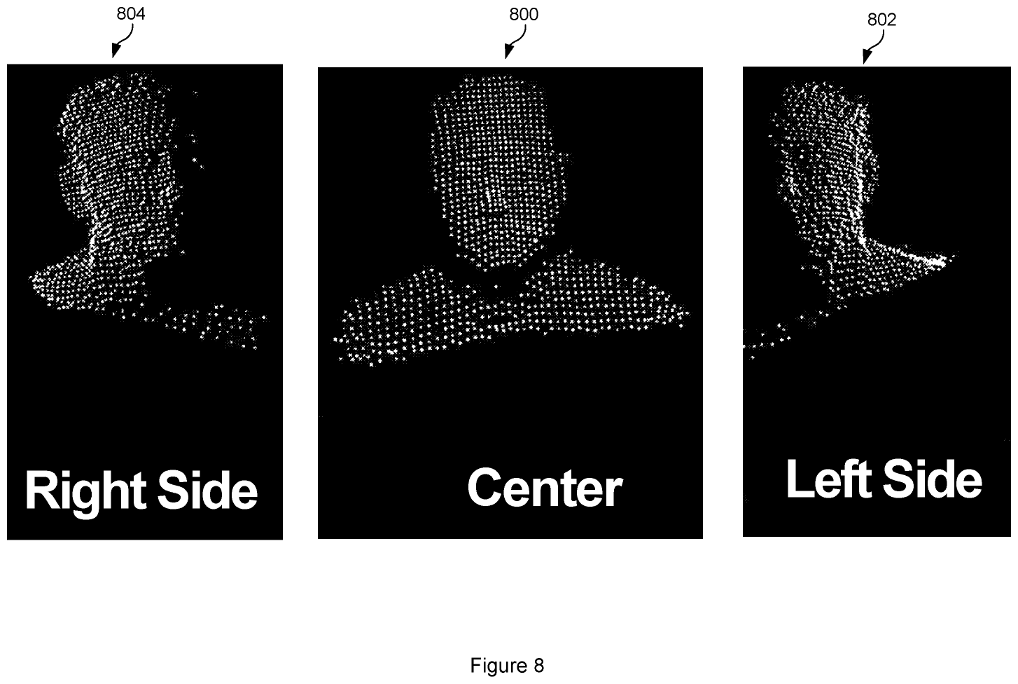

FIG. 9 is a representation of merged, multi-perspective point cloud data.

FIG. 10 is an illustration of one embodiment of a grid overlaying an imaging area.

FIG. 11 is an illustration one of embodiment of points from a point cloud organized in a grid.

FIG. 12 is an illustration of one embodiment of formation of a polygonal mesh based on point cloud data.

FIG. 13 is an illustration of generation of normal vectors based on portions of the polygonal mesh.

FIG. 14 is an illustration of one embodiment of a reconstructed surface with waypoints and spray vectors.

FIG. 15 is an illustration of one embodiment of a treatment path.

### DETAILED DESCRIPTION

Cooling based treatments are frequently used to address a wide range of health and aesthetic issues. These issues can include, for example, the ablation of benign lesions such as, for example, acne-vulgaris, cystic; acne keloidalis; adenoma sebaceum; alopecia areatea; angiokeratomas; angiokeratoma of Fordyce; atypical fibroxanthoma; cherry angiomas; chonrodermatitis nodularis helicis; chromoblastomycosis; clear cell acanthoma; condyloma acuminatum; dermatofibroma; disseminated superficial actinic porokeratosis; elastosis perforans serpiginosa; epidermal nevus; erosive adenomatosis of the nipple; folliculitis keloidalis; granuloma annulare; granuloma faciale; granulomaa pyogenicum; hemangioma; herpes labialis; idiopathic guttate hypomelanosis; Kyrle's disease; leishmaniasis; lentigines; lentigo simplex; lichen sclerosus et atrophicus of vulva; lupus erythematosus; lymphangioma; lymphocytoma cutis; molluscum contagiosum; mucocele; myxoid cyst; orf; porokeratosis plantaris discreta; porokeratosis of Mibelli; prurigo nodularis; pruritus ani; psoriasis; rhinophyma; rosacea; sarcoid; sebaceous hyperplasia; seborrheic keratosis; solar lentigo; syringoma; trichiasis; trichoepithelioma; varicose veins; venous lakes; verrucae-periungual, plane, vulgaris, filiform, plantar; xanthoma; acne scar; keloids; cutaneous horn; hypertrophic scar; ingrown toenail; skin tags; tattoos; freckles; spider naevus; capillary haemangioma; cavernous haemangioma; milia; trichillemmal cyst; steatocystoma multiplex; hidrocystoma; acrokeratosis veruciformis; dermatosis papulose nigra; hyperkeratosis naevoid of nipple; benign lichenoid keratosis; angiofibromas; and angiomas. In some embodiments, cooling based treatments can be used to treat pre-malignant skin conditions such as, for example: actinic keratosis; leukoplakia; Bowen disease; erythroplasia of Quyrat; keratoacanthoma; and lentigo maligna, and can be used to treat malignant skin conditions such as, for example: basal cell carcinoma; Kaposi sarcoma; squamous cell carcinoma; and melanoma.

Some of these treatments have been specifically designed to cause skin healing and/or to change a color of the skin via the creation of skin lightening or of skin darkening. This color change of the skin may be localized to a small skin area, or may affect a large area of skin. The area of to be treated skin can make such treatment difficult as adequate consistency of treatment may be difficult to achieve. These treatments can include cooling treated skin to specific temperatures and/or temperature ranges, and in some instances can include maintaining those temperatures and/or temperature ranges for a predetermined time and/or range of times. In some instances, the effectiveness of many treatments is dependent on the providing of specific amounts of cooling for specific amounts of time. Further, the difficulty in achieving consistent results increases as the treated area increases.

The present disclosure relates to systems, devices, and methods that improve the planning and/or delivery of a treatment. In some embodiments, this can include the delivery of a treatment to: change a color of the skin such as by causing skin lightening or darkening; ablate a lesion; and/or facilitate skin healing. In some embodiments, delivery of a treatment can include, for example: applying a cryotherapy to the skin; applying electromagnetic energy to the skin; applying one or several lasers or laser beams to the skin; and/or applying a substance to the skin such as, for example, a medication, a pigment, a dye, a paste, and/or an ink. In some embodiments, the application of one or several of these treatments can be alternative or adjunctive to others of these treatments.

This improved planning and/or delivery of the treatment can be achieved by a system and/or by use of a system that includes a cryospray applicator coupled to a distal end of a mechanical arm that can be a multi-axis arm. The position and/or orientation of the cryospray applicator can be controlled by movement of the mechanical arm and/or by movement of one or several joints of the mechanical arm. The mechanical arm can be controlled to sweep the cryospray applicator across the patient's skin to treat a desired area of skin. The sweeping of the cryospray applicator can be controlled according to information received from one or several of the sensors including, for example, the temperature of the skin, the distance between the cryospray applicator and the skin being treated, and/or the orientation of the cryospray applicator with respect to the skin.

The cryospray applicator can include one or several sensors that can detect, for example, a distance between the cryospray applicator and the skin being treated, the orientation of the cryospray applicator with respect to the skin being treated, and/or the cooling or temperature of the skin being treated. The cryospray applicator can include a visualization system that can generate images of the patient and/or of portions of the patient before and/or during a treatment. In some embodiments, the visualization system can include a Time-of-flight camera and/or an infrared camera. The cryospray applicator can further include a nozzle control, which nozzle control can change nozzles of the cryospray applicator to affect a size of treatment footprint of the cryospray applicator to provide a desired size of the treatment footprint. Nozzles can be changed to change the size of the treatment footprint to facilitate treatments of small skin areas and/or to provide improved dosing control.

The system can include a controller which can control the operation of the mechanical arm, the cryospray applicator, the sensors, the visualization system, and/or the nozzle control. The controller can receive information relating to the patient and the area of the patient's skin to be treated and can generate a treatment plan for the patient. The generation of the treatment path can include the generation of one or several point clouds representing the topography of the portion of the patient to be treated. These one or several point clouds can be generated by a Time-of-flight camera that can be coupled to the cryospray applicator. The one or several point clouds can be processed to generate a surface representative of the portion of the patient to be treated. This can comprise the formation of a polygonal mesh. From the polygonal mesh, surface-normal vectors can be calculated and then combined to determine delivery vectors. Waypoints can be added along each of these delivery vectors at a desired distance from the portion of the patient to be treated by the cryospray applicator providing treatment along that delivery vector.

Once waypoints and delivery vectors have been identified, waypoints and delivery vectors can be arranged to form one or several treatment paths. In some embodiments, these paths can be created by linking adjacent waypoints in a pattern snaking through the grid of waypoints and delivery vectors. In some embodiments, these paths can be created by linking adjacent waypoints in a direction which can be preselected, or selected based on a user input. In some embodiments, waypoints can be linked according to an evaluation of one or several potential treatment paths. In some embodiments, this can include the creation of a plurality of potential treatment paths, and identifying and selecting the one of the plurality of potential treatment paths that is and/or is identified as a best treatment path. In some embodiments, this best treatment path can be the treatment path that requires the least movement of the cryospray applicator, or in other words, that has the smallest aggregate difference between delivery vectors of adjacent waypoints in the treatment path.

Delivery of treatment along the delivery vector and from the waypoints can improve the effectiveness and consistency of delivered treatments. Specifically, maintaining a constant angle between the delivery of treatment and a treated surface maintains an even distribution of treatment across the treatment footprint, and maintain a constant footprint size. Specifically, changing of the angle between the delivery of treatment and the treated surface changes the size of the footprint and thus changes the concentration of treatment administered to the treated surface. Similarly, maintaining constant distance between the cryospray applicator and the treated surface maintains a constant treatment footprint, and thus maintains a constant concentration of administered treatment. The use of waypoints and delivery vectors provide effective control of distance between the cryospray applicator and the treated surface and the angle between the delivery of treatment and the treated surface. This can improve consistency of treatment and can improve clinical outcomes.

In some embodiments, these treatments can be affected by the identification of patient features, the ascertaining of one or several attributes of the patient's skin, or the like. The controller can direct operation of all or portions of the system to ascertain one or several attributes of the patient and/or of the patient's skin. This can include generating images of the patient and/or of the area of the patient's skin to be treated, ascertaining underlying skin structure of all or portions of the area of the patient's skin to be treated, and/or measuring perfusion of the skin and or the thermal response of the skin to cooling. In some embodiments, this can include identifying one or several keep-out zones corresponding to portions of the patient to which no treatment is delivered. Examples of keep-out zones include, for example, eyes, nasal passages, ear canals, or the like. The treatment plan can be used to control and/or direct the delivery of treatment to the patient. In some embodiments, the treatment plan may remain constant, and in some embodiments, the treatment plan can be modified as the treatment is being delivered.

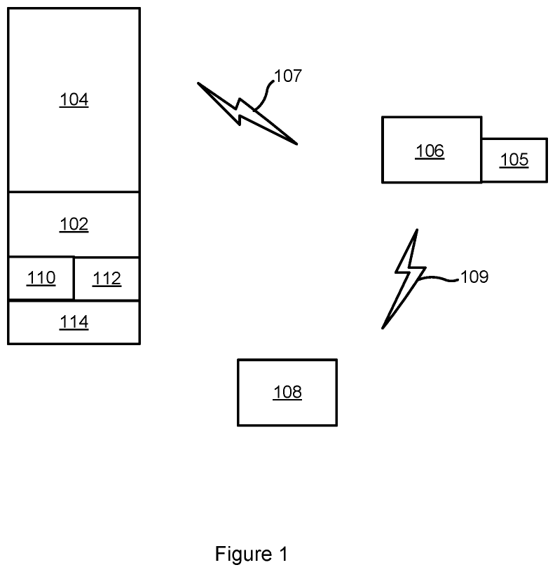

With reference now to FIG. 1, a schematic illustration of one embodiment of a skin cooling treatment system 100 is shown. The skin cooling treatment system 100 can include a cryospray applicator 102 that is coupled to a mechanical arm 104, and specifically to a distal end of the mechanical arm 104. The cryospray applicator 102 can be configured to deliver a coolant to a treated portion of skin. In some embodiments, the cryospray applicator 102 can be configured to deliver a spray of cryogen towards and/or or onto a portion of skin being treated. This spray of cryogen can be delivered through one or several orifices, which orifices can comprise one or several nozzles. Embodiments of an exemplary cryospray applicator 102 including an array of orifices are disclosed in U.S. application Ser. No. 16/020,852, filed on Jun. 27, 2018, and entitled, “Dermatological Cryospray Devices Having Linear Array Of Nozzles And Methods Of Use”, the entirety of which is hereby incorporated by reference herein. Further details of the mechanical arm 104, the cryospray applicator 102, and controlling the same can be found in U.S. application Ser. No. 16/723,633, filed on Dec. 20, 2019, and entitled “AUTOMATED CONTROL AND POSITIONING SYSTEMS FOR DERMATOLOGICAL CRYOSPRAY DEVICES,” and U.S. application Ser. No. 16/723,859, filed on Dec. 20, 2019, and entitled “AUTOMATED DERMATOLOGICAL CRYOSPRAY TREATMENT PLANNING SYSTEM,” the entirety of each of which is hereby incorporated by reference herein.

The mechanical arm 104 can have any desired number of axes of movement, and can, in some embodiments, be a 6-axis arm. In some embodiments, the mechanical arm 104 can have a single degree of freedom (e.g. a linear stage) which would allow control of movement along one axis, two degrees of freedom which would allow control of movement along two axes, three degrees of freedom, four degrees of freedom, five degrees of freedom, six degrees of freedom, and/or any other number of degrees of freedom. In some embodiments, the number of degrees of freedom can be selected based on the desired level of control and movement of the cryospray applicator. Thus, a higher number of degrees of freedom provide greater control of the position and/or orientation of the cryospray applicator 102. The mechanical arm 104 can be any of a number of currently commercially available mechanical arms. The mechanical arm 104 can be robotic and/or teleoperated.

The system 100 can include a controller 106 and/or processor 106 which can be communicatively coupled with the mechanical arm 104 and specifically with one or several actuators in the mechanical arm 104. In some embodiments, the communicating coupling of the controller 106 and the mechanical arm 104 can be via a wired or wireless connection, and the communicating coupling is indicated by lightning bolt 107. The processor 106 can comprise a microprocessor, such as a microprocessor from Intel® or Advanced Micro Devices, Inc.®, or Texas Instrument, or Atmel, or the like.

The controller and/or processor 106 can be communicatingly coupled with a memory, which memory can be volatile and/or non-volatile and/or can include volatile and/or non-volatile portions. In some embodiments, the memory can include information relating to one or several patients, one or several planned treatments, and/or one or several delivered treatment. The memory relating to one or several patients can include, for example, a unique patient profile associated with each patient, and/or a unique provider profile associated with each provider. In some embodiments, a patient's patient profile can include information identifying one or several attributes of the patient including, for example, the patient's medical history, the patient's treatment history including, for example, information relating to one or several treatments provided to the patient, and/or information relating to the efficacy of one or several previously provided treatments. In some embodiments, the provider profile can include information relating to treatments provided to the provider's patients and/or the effectiveness of these provided treatments.

The memory 105 can include the information relating to one or several planned treatments. This information can include, for example, all or portions of information used in delivering a treatment. This can include, for example, information relating to one or several treatment paths, height and/or orientation specifications, dosing information, or the like. The memory 105 can further include a database with information relating to treatment results. This information can, for example, identify treatment effectiveness, information relating to one or several responses associated with a treatment, or the like. In some embodiments, this information can be specific to one or several patients and can be linked with the one or several patient profiles of those one or several patients.

The controller 106 and/or processor 106 can generate a treatment plan and can generate control signals which can control the movement of the cryospray applicator 102 according to the treatment plan. In some embodiments, the treatment plan can remain constant during the treatment, and in some embodiments, the treatment plan can be adjusted as the treatment is being provided. The control of the movement of the cryospray applicator 102 can allow the processor 106 to control: the sweeping of the cryospray applicator 102 across the patient's skin; the distance between the cryospray applicator 102 and the portion of skin being presently treated; and/or the orientation of the cryospray applicator 102 with respect to the portion of skin being presently treated.

The controller 106 can, in some embodiments, receive information relating to the desired area of skin for treatment and information relating to the treatment. With this information, the controller 106 can, in some embodiments, generate treatment paths, which treatment paths characterize the movement of the cryospray applicator 102 and the delivery of cooling the cryospray applicator 102. In some embodiments, the controller 106 can change these treatment paths during the providing of a treatment. In some embodiments, for example, the size of the portion of skin treated at any instant by the cryospray applicator 102 may vary based on, for example, the nozzle being used to deliver the treatment, the number of orifices in the array of orifices through which cryogen is sprayed, the distance between the portion of skin being treated and the cryospray applicator 102, or the like. In such embodiments, as the size of the portion of skin treated at any instant changes, the controller 106 can generate updated treatment paths to compensate for this change in the size of the portion of skin treated at any instant.

The controller 106 can be communicatingly connected with a user device 108. The user device can be distinct from the controller 106, or in some embodiments, the user device 108 can include the controller 106. The user device 108 can be any device configured to provide information to and receive inputs from a user, such as the user controlling the treatment provided by the skin cooling treatment system 100. The user device 108 can, in some embodiments, comprise a computing device such as a laptop, a tablet, a smartphone, a monitor, a display, a keyboard, a keypad, a mouse, or the like. In some embodiments, the communicating coupling of the controller 106 and the user device 108 can be via a wired or wireless connection, and the communicating coupling is indicated by lightning bolt 109.

The cryospray applicator can include a sensing subsystem 110, a visualization subsystem 112, and/or a nozzle control 114. The sensing subsystem 110 can include a plurality of sensors 206. These sensors can include a plurality of sensors that can be configured to detect and/or determine a distance between the cryospray applicator 102 and/or an orientation of the cryospray applicator 102 with respect to the patient's skin, and specifically with respect to an instantaneous treatment footprint. The visualization subsystem 112 can comprise one or several cameras. These one or several cameras can comprise one or several cameras configured to generate image data, also referred to herein as imagery. The generated image data can include image data in the visible spectrum and/or image data in the non-visible spectrum. As used herein, “image data” can be any type of data generated by one or several cameras such as in the visualization system 112, this data including, for example, 2-D image data and/or 3-D image data. In some embodiments, the 2-D and/or 3-D image data can be still or video data. In some embodiments, the 3-D image data can include point-cloud data. The nozzle control 114 can identify a current nozzle used by the cryospray applicator, can identify a desired treatment footprint, and can select a next nozzle that best achieves the desired treatment footprint.

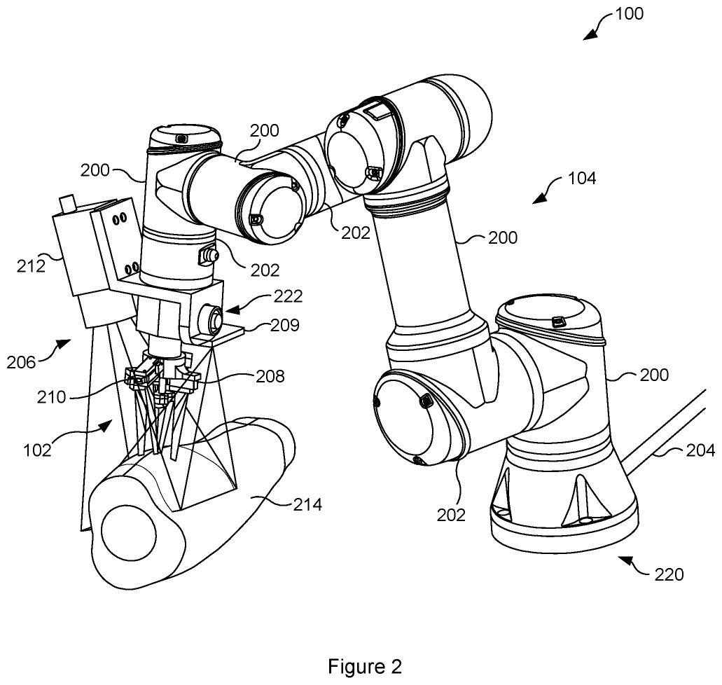

With reference now to FIG. 2, a perspective view of one embodiment of the skin cooling treatment system 100 is shown. The system includes the cryospray applicator 102 and the mechanical arm 104. As seen in FIG. 2, the mechanical arm 104 comprises a plurality of linkages 200 coupled by a plurality of joints 202, which joints 202 allow the relative movement of the linkages 200 with respect to each other. The mechanical arm 104 can further include a plurality of actuators, which actuators can, in response to control signals received from the controller 106, affect the relative position of some or all of the linkages 200 via movements of some or all of the joints 202 coupling linkages 200 to thereby affect the position and/or orientation of the cryospray applicator 102.

The mechanical arm 104 can further include one or several communication features such as cable 204. In some embodiments, the communication features, such as the cable 204 can communicatingly couple the mechanical arm 104, and specifically the actuators of the mechanical arm 104, to the controller 106.

The mechanical arm 104 further comprises a proximal end 220 and a distal end 222. In some embodiments, the proximal end 220 of the mechanical arm 104 can be secured to an object such as, for example, a floor, a table, a cart, a wagon, or the like. The distal end 222 of the mechanical arm 104 can be coupled to the cryospray applicator 102 and can move with respect to the proximal end 220 of the mechanical arm 104. In some embodiments, the processor 106 can be configured to control the distal end 222 of the mechanical arm 104 and/or to control the cryospray applicator 102.

The cryospray applicator 102 can include a plurality of sensors 206, which sensors can include one or several alignment sensors 208, one or several time-of-flight (“TOF”) cameras 209, one or several distance sensors 210, and/or one or several temperature detection features 212. In some embodiments, the sensors 208, 209, 210, 212 belong to the sensing subsystem 110. These sensors 206 can, in some embodiments, sense information relating to the treatment of a patient 214, and specifically to the treatment of some or all of the patient's skin.

The TOF camera 209 can be a range imaging camera. In some embodiments, and as shown in FIG. 2, the TOF camera can be coupled to the cryospray applicator 102 and/or to the mechanical arm 104. The TOF camera 209 can, in some embodiments, achieve this range imaging via use of time-of-flight techniques to resolve distance between the camera and the subject for each point of the image. This can include measuring the round trip time of an artificial light signal provided by a laser or an LED incorporated in or controlled by the TOF camera 209.

The TOF camera 209 can, in some embodiments, determine distances between the TOF camera 209 and surfaces in an imaging area. The TOF camera 209 can determine the distance to surfaces in the imaging area, which, when the TOF camera 209 is positioned over the patient, or over a portion of the patient's body, can include the patient and/or a portion of the patient's body. Thus, the TOF camera 209 can determine the distance to the surface of the patient's body and/or to the surface of portions of the patient's body in the imaging area. The TOF camera 209 can generate a point cloud, a set of data points in space, each of the points represents a location of a portion of a surface in the imaging area with respect to the camera.

In some embodiments, the TOF camera 209 can be used in addition to other sensors such as, for example, in addition to one or several alignment sensors 208, one or several distance sensors 210, and/or one or several temperature detection features 212. In some embodiments, the inclusion of the TOF camera 209 can allow the elimination of one or several of the alignment sensors 208, the distance sensors 210, and/or the temperature detection features 212. In some embodiments, for example, the sensing subsystem 110 can include the one or several TOF cameras 209 and the one or several temperature detection features 212.

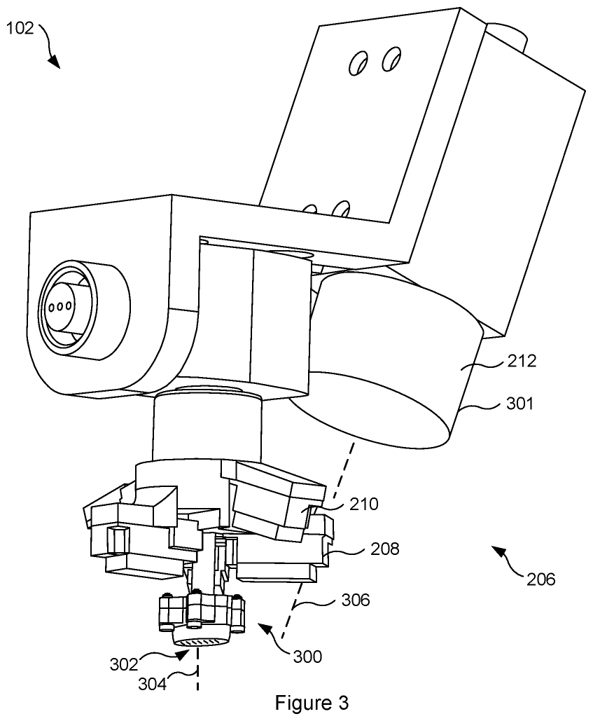

With reference now to FIG. 3, a perspective view of one embodiment of the cryospray applicator 102 is shown, which cryospray applicator 102 can be coupled to the distal end 222 of the mechanical arm 104. The cryospray applicator 102 includes a spray head 300 which comprises an array of orifices 302 through which cryogen can be sprayed towards and/or onto the patient's skin and specifically towards and/or onto a portion of the patient's skin being presently treated.

In some embodiments, the cryospray applicator 102 comprises the plurality of sensors 206, and specifically comprises one or more of: one or several alignment sensors 208; one or several distance sensors 210; or one or several temperature detection features 212. In some embodiments, the one or several temperature detection features 212 can be configured to: detect freezing of the portion of the patient's skin being presently treated; detect a temperature of the portion of the patient's skin being presently treated; detect a freezing rate of the portion of the patient's skin being presently treated, or the like. In some embodiments, the temperature detection feature can comprise a camera, and specifically can comprise an infrared camera 301. In some embodiments, the infrared camera 301 can be pointed at the portion of the patient's skin being presently treated, or in other words, an axis 304, also referred to herein as “the line of spray 304”, centrally extending through the array of orifices 302 intersects with an axis 306 central to the field of view of the infrared camera 301 such that the portion of skin being presently treated is within the field of view of the infrared camera 301. In embodiments in which the one or several temperature detection features 212 comprises one or several cameras, the one or several temperature detection features 212 can belong to the visualization subsystem 112.

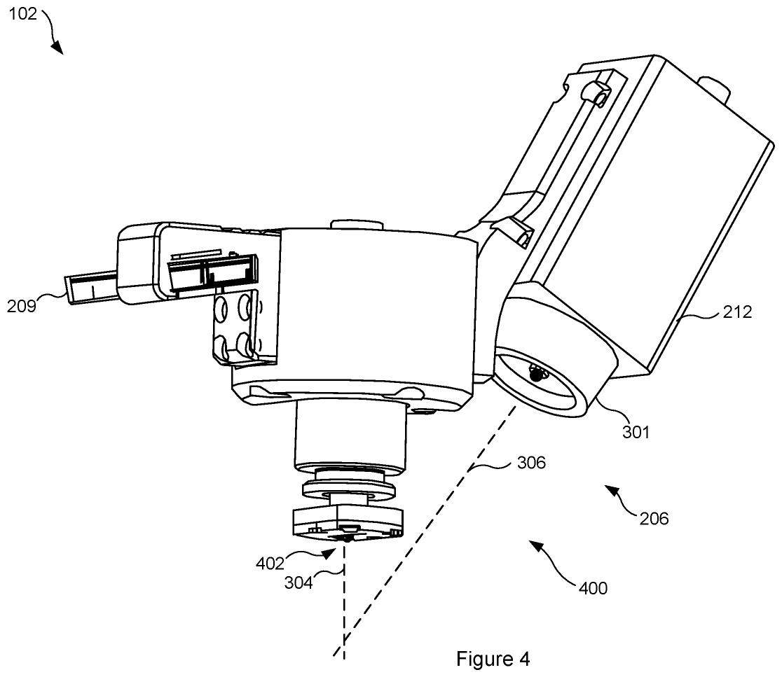

With reference now to FIG. 4, a perspective view of one embodiment of the cryospray applicator 102 is shown, which cryospray applicator 102 can be coupled to the distal end 222 of the mechanical arm 104. The cryospray applicator 102 includes a spray head 400 which comprises an array of orifices 402 through which cryogen can be sprayed towards and/or onto the patient's skin and specifically towards and/or onto a portion of the patient's skin being presently treated.

In some embodiments, the cryospray applicator 102 comprises the plurality of sensors 206, and specifically comprises one or more of: one or several TOF cameras 209; and/or one or several temperature detection features 212. In some embodiments, the one or several temperature detection features 212 can be configured to: detect freezing of the portion of the patient's skin being presently treated; detect a temperature of the portion of the patient's skin being presently treated; detect a freezing rate of the portion of the patient's skin being presently treated, or the like. In some embodiments, the temperature detection feature can comprise a camera, and specifically can comprise the infrared camera 301. In some embodiments, the infrared camera 301 can be pointed at the portion of the patient's skin being presently treated, or in other words, the line of spray 304, centrally extending through the array of orifices 302 intersects with the axis 306 central to the field of view of the infrared camera 301 such that the portion of skin being presently treated is within the field of view of the infrared camera 301.

In some embodiments, the TOF camera 209, as shown in FIG. 1 can be pointed at the treatment area. In such embodiments, at least the portion of skin being presently treated can be within the field of the TOF camera 209, and in some embodiments, the fields of view of the TOF camera 209 and the of the infrared camera 301 can overlap, or at least partially overlap, as shown in FIG. 1. In some embodiments, the TOF camera 209 can belong to the visualization subsystem 112.

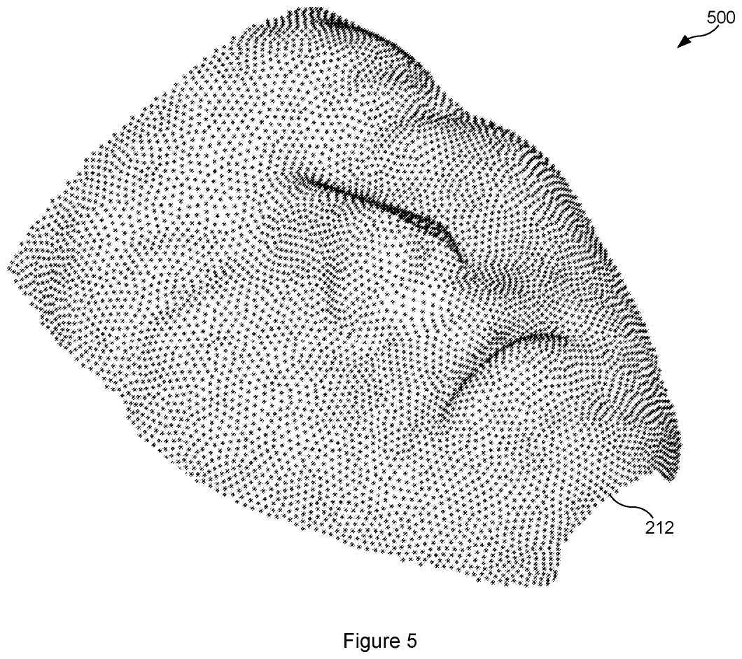

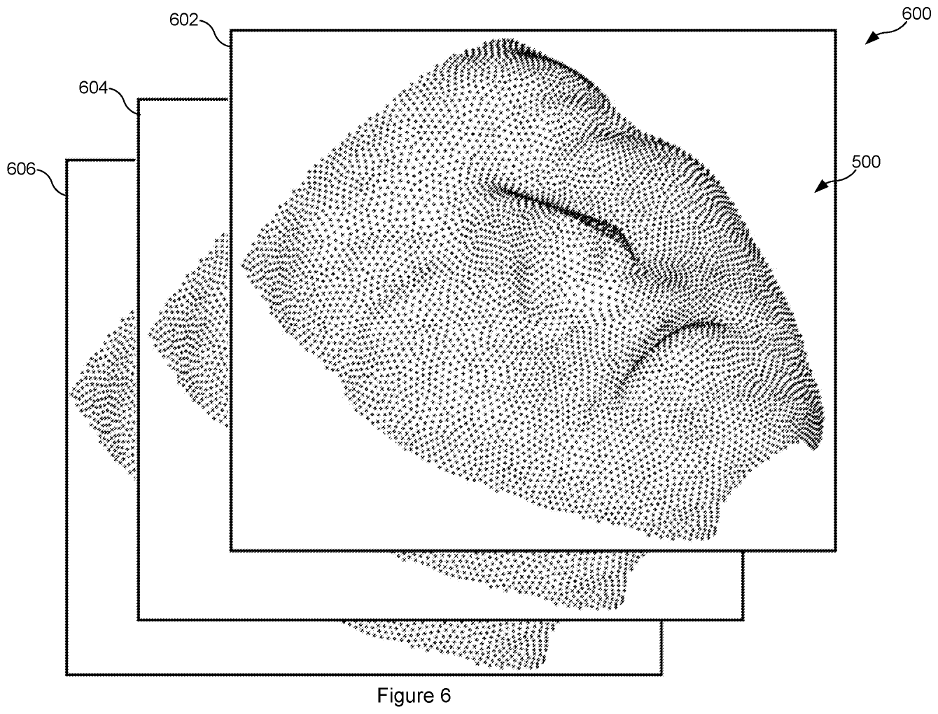

With reference now to FIG. 5, an illustration of a point cloud 500 representing a face is shown. The point cloud 500 can comprise a plurality of points, each representing a location in space. In some embodiments, this location in space can be with respect to the TOF camera 209. The TOF camera 209 can generate frames, each frame comprising a point cloud 500. FIG. 6 shows a plurality of frames 600, each of the frames comprising a point cloud 500. The frames 600 include a first frame 602, a second frame 604, and a third frame 606. The TOF camera 209 can generate frames 600 at a frame rate. This frame rate can be, for example, 2 Hz, 5 Hz, 10 Hz, 20 Hz, 50 Hz, 100 Hz, 200 Hz, 500 Hz, between 1 and 100 Hz, between 2 and 50 Hz, between 5 and 20 Hz, and/or any other or intermediate value or between any other or intermediate range.

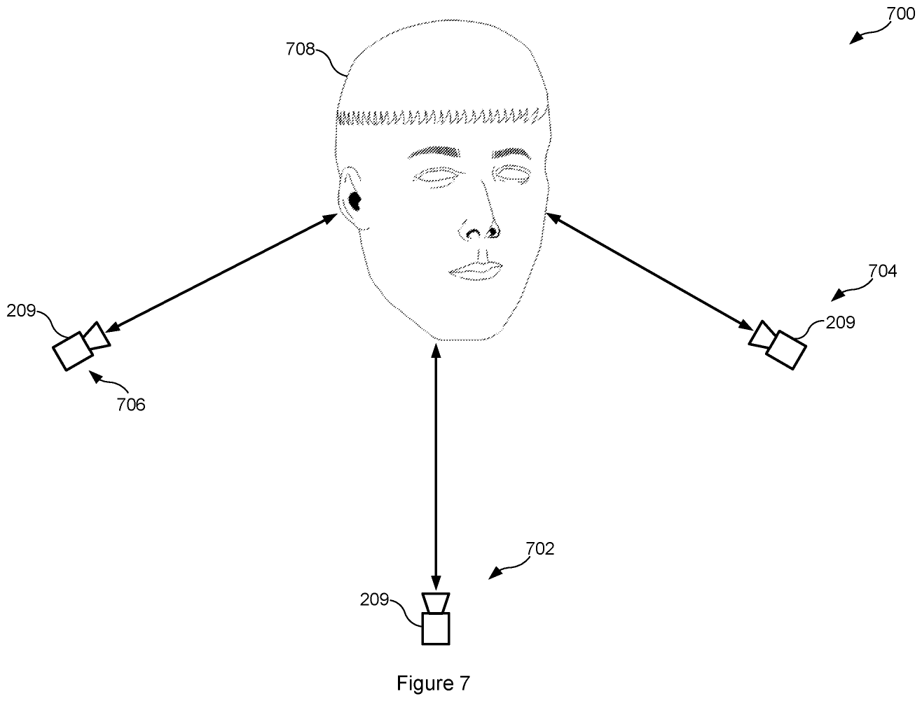

In some embodiments, the point cloud 500 can comprise a merged point cloud, also referred to herein as a 3D cloud. The merged point cloud can be created via the merging of point clouds generated via imaging of the same object from different perspectives. FIG. 7 depicts one embodiment of multi-perspective point cloud generation 700. As seen in FIG. 7, a camera 209 is moved between a central position 702, a left position 704, and a right position 706, and generates a point cloud of the imaged object 708 from each of these positions. Alternatively, a different camera 209 can be located in each of the positions 702, 704, 706, and each camera 209 can generate a point cloud from their respective position 702, 704, 706.

The positions 702, 704, 706 have a known offset with respect to each other, and are a known distance from the imaged object 708. In some embodiments, for example, each of the positions 702, 704, 706 are the same distance from the imaged object 708, but are at different or angular positions with respect to the imaged object 708. Thus, from the perspective of the imaged object 708, the position 704 is at some negative angle, or in other words some negative angular offset from position 702, and position 706 is at some positive angle, or in other words some positive angular offset from position 702. In some embodiments, the positions 704, 706 can have the same angular offset from position 702. This angular offset can be, for example, 5 degrees, 10 degrees, 20 degrees, 30 degrees, 45 degrees, 60 degrees, 75 degrees, 90 degrees, or any other or intermediate angle. In some embodiments, this angular offset can be, for example, between 10 and 90 degrees, between 30 and 70 degrees, between 40 and 50 degrees, or between any other or intermediate angles.

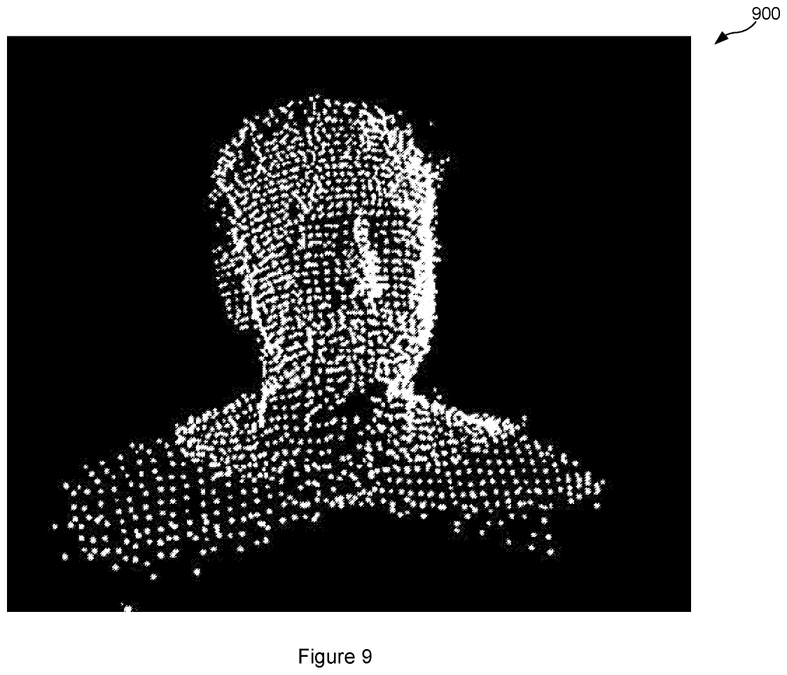

Generating of point clouds 500 from the positions 702, 704, 706 can result in the generation of a center point cloud 800 by the camera 209 at position 702, a left side point cloud 802 by the camera 209 at position 704, and a right side point cloud 804 by the camera 209 at position 706. In some embodiments, these point cloud 800, 802, 804 can be combined into a single merged point cloud 900, such as is shown in FIG. 9. In some embodiments, the formation of the single merged point cloud 900 can include the location of each of the point clouds 800, 802, 804 with a common orientation in a common space. In some embodiments, this includes moving the point clouds 800, 802, 804 to the position of the imaged object. Specifically, this can include, for each of the point clouds 800, 802, 804 subtracting the distance from the imaged object 708 to the camera generating the point cloud 800, 802, 804, for the left side point cloud 802 and the right side point cloud 804, and rotating the point cloud 802, 804 in a direction and amount opposite to the angular offset used to create that point cloud 802, 804. Having eliminated the distance and the angular offset, the point clouds 800, 802, 804 are in the same 3D space and are combined into a single array containing the points of all of the point clouds to thereby create the merged point cloud 900. The merged point cloud 900 is a subset of point cloud 500, and thus, as used herein, a point cloud 500 can include the merged point cloud 900.

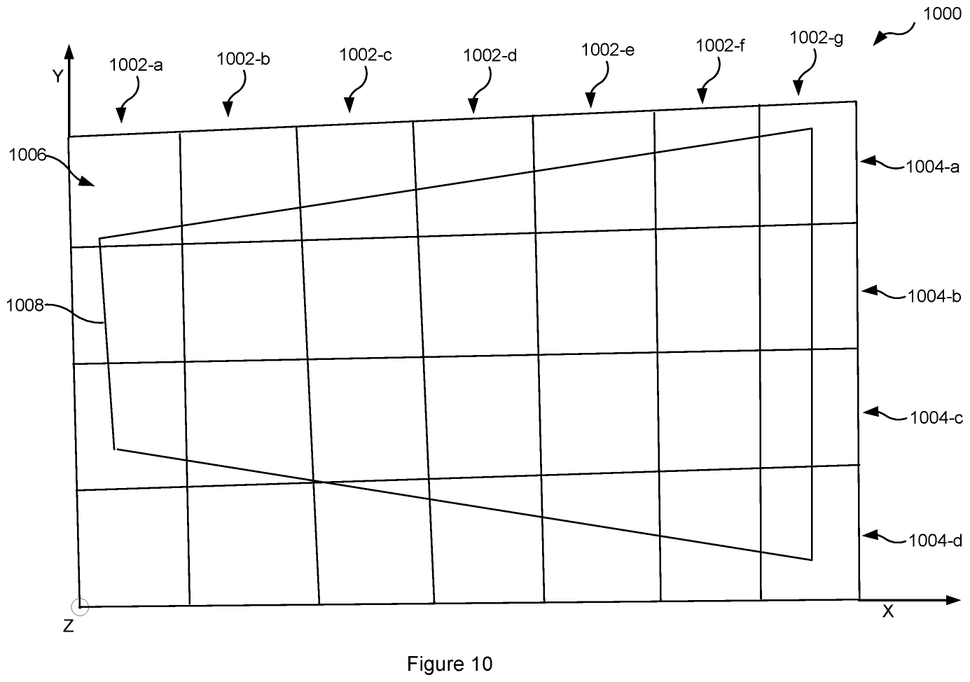

The point clouds 500 generated by the TOF camera 209 can be noisy and in some embodiments, non-uniform. In some embodiments, these point clouds 500 can be noisy and/or non-uniform in that points in the point cloud 500 can be unequally spaced and/or in that adjacent points within the point cloud 500 can vary in a manner not reflective of changes to the surface they are detecting. Similarly, differences can arise between frames 600 of point clouds, which differences reflect noise. Further, a point cloud 500 and/or frames 600 of point clouds 500 can include so many points as to complicate processing and/or use of the points 502. FIGS. 10-15 illustrate steps in one embodiment of a process for using one or several point clouds to generate movement paths for controlling operation of a cryospray applicator 102. These movement paths can include waypoints and delivery vectors. As used herein, a waypoint is a location in space that the cryospray applicator 102 should be moved to, or through during a treatment. These waypoints can, in some embodiments, specify a treatment area for delivery of a treatment.

In some embodiments, the TOF camera 209 outputs an unorganized point cloud, which unorganized point cloud can comprise a list of points, and specifically, a flat list or array of points. Points from the point cloud can be sorted into a grid. FIG. 10 shows one embodiment of a 2D grid 1000 that can include a plurality of columns 1002 and rows 1004 of boxes 1006. The rows 1002, columns 1004, and/or boxes can, in some embodiments, be uniformly sized and shaped. The grid 1000 be used as an overlay on the imaging area, the area imaged by the TOF camera 209, as represented by the overlaying of the grid on imaged object 1008. The grid 1000 extends in two directions (X and Y directions) within the plane of the imaging area and in a third direction (Z direction) perpendicular to the plane of the imaging area. In some embodiments, the boxes 1006 of the grid 1000 can comprise rectangular prisms extending infinitely in the Z direction. In some embodiments, the grid 1000 can comprise a 3D grid comprising a plurality of voxels or cubes.

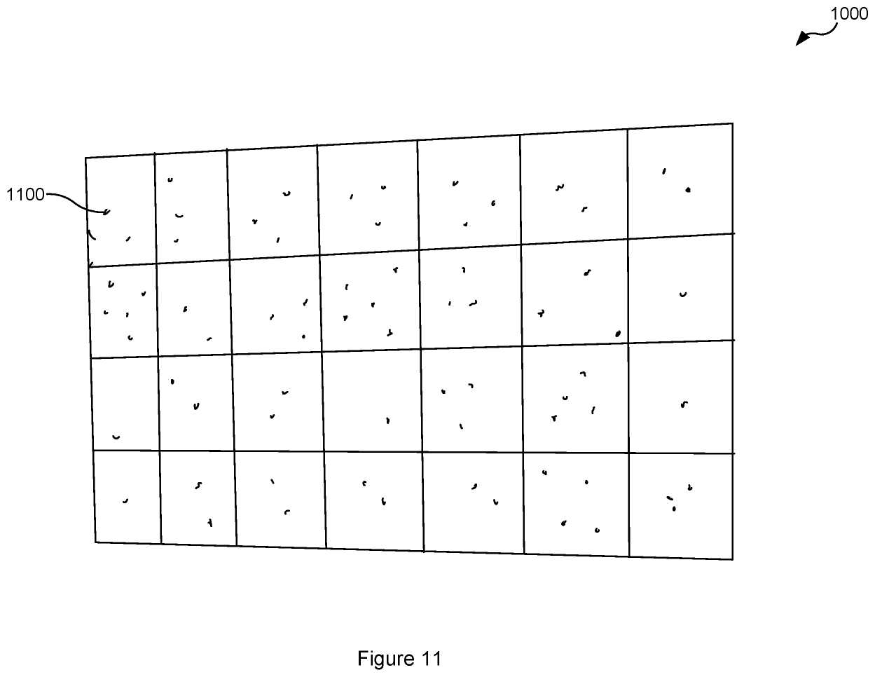

The points 1100 of the point cloud can be organized into the grid 1000 as indicated in FIG. 11. Specifically each point 1100 in the point cloud can be placed in the box 1006 corresponding to the location of that point 1100 in the plane of the imaging area. Because of non-uniformities in the point cloud, boxes may have different numbers of points 1100, or in other words, the distribution of points 1100 across the grid 1000 can be uniform or non-uniform. In embodiments in which a merged point cloud 900 is used, the merged point cloud can be organized into a 3D grid.

Each point 1100 in the point cloud 500 can include location information identifying a location of the point with respect to the plane of the imaging area as well as the distance of the point 1100 from the TOF camera 209 and/or from the plane of the imaging area. This distance of the point 1100 from the TOF camera 209 and/or from the plane of the imaging area is referred to herein as “depth.” For a 2D grid, placing the point 1100 in the box corresponding to the location of the point in the plane of the imaging area does not affect the depth of that point 1100 perpendicular to the plane of the imaging area. Thus, in an embodiment in which a box 1006 contains multiple points 1100, some or all of those multiple points 1100 can have a different depth. In some embodiments, points from a point cloud from a single frame can be organized into the grid 1000, and in some embodiments, point clouds from multiple frames can be organized into a single grid 1000.

For a 3D grid, the placing of each point in the point cloud 500 in the grid includes identifying the voxel including the X, Y, and Z location of that point in the point cloud 500. After this voxel is identified, a representation of the point is created within the voxel. In some embodiments, this representation of the point can be created at the actual location within the voxel.

The points 1100 in each box 1006 can be resolved into a single point. Resolving the points 1100 in a box 1006 into a single point can include determining an average depth for all of the points 1100 in the box 1006 and applying this average depth to the single point. In some embodiments, this single point can be placed at the center of the box 1006 or voxel, and in some embodiments, this single point can be placed at the average location within the box 1006 or voxel of the points 1100 within the box 1006. Determination of the average depth for points 1100 in the box 1006 or voxel facilitates in eliminating and/or mitigating noise in the point cloud. Further, representing multiple points 1100 from the point cloud with a single point per box 1006 or voxel simplifies the point cloud. Further, resolving the unorganized point cloud to a single point per box or voxel in a uniform grid can facilitate further operations performed on those points. Information for the point of each box or voxel can be stored.

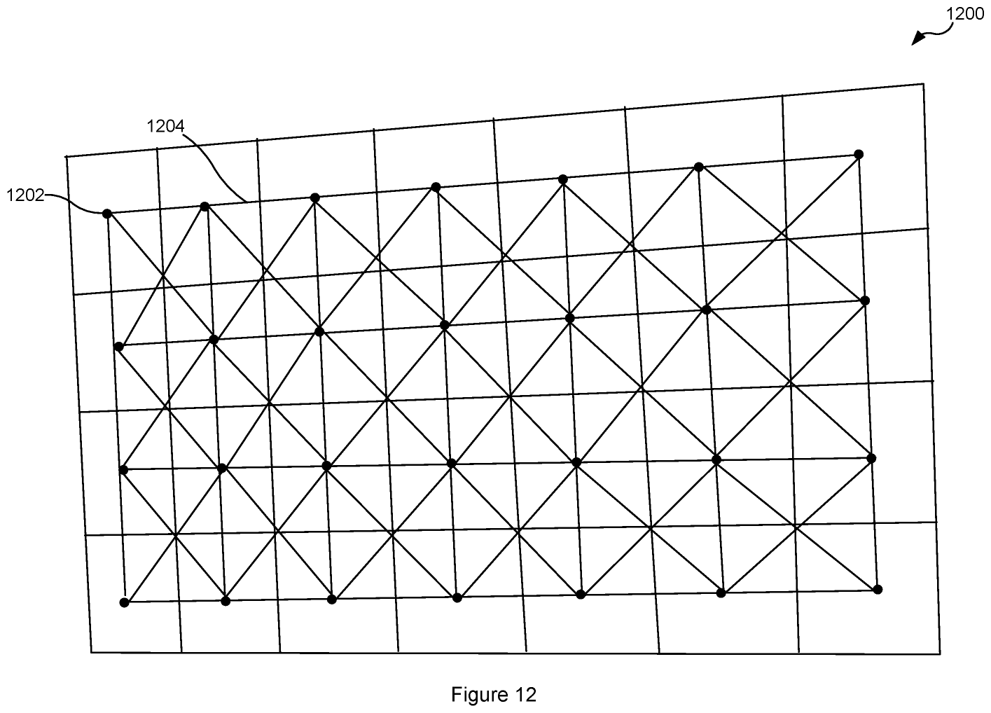

Having resolved the points 1100 in each box 1006 or voxel to a single point, these single points are used to form a polygon mesh 1200 as shown in FIG. 12. Specifically, these single points, also referred to herein as vertices 1202, are linked to form a polygon mesh by edges 1204 that are created and that link these vertices. In some embodiments, edges 1204 are created linking neighboring vertices, also referred to herein as adjacent vertices. These edges 1204 thus each link a pair of vertices 1202, and combinations of these edges can form polygons. In some embodiments, if a vertex is adjacent one or several boxes or voxels that do not have a vertex, then these boxes or voxels can be skipped over. In some embodiments, rules can identify the maximum number of boxes or voxels that can be skipped and still create an edge. In some embodiments, this maximum number can be, for example, 1, 2, 3, 4, 5, 10, 15, 20, 50, or any other or intermediate number of boxes or voxels.

In some embodiments, the forming of this polygonal mesh 1200 can comprise the formation of a triangle mesh, a quad mesh, or an n-gon mesh. The creation of the polygon mesh 1200 creates a geometric model of the surface in the treatment area. Edge information, and information relating to the created polygon mesh can be stored.

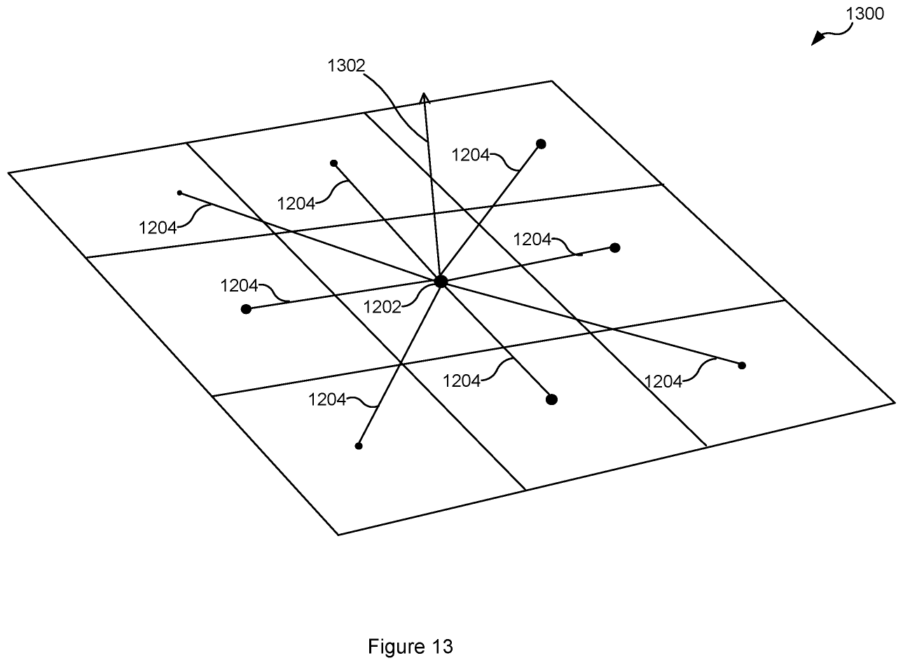

FIG. 13 depict one embodiment of the creation 1300 of a normal vector 1302 for a vertex 1202. After the creation of edges 1204 and/or of the polygon mesh 1200, a normal vector 1302 can be generated for each vertex 1202 in the polygon mesh 1200, which normal vector 1302 can be normal to the surface at the location of the vertex 1202. In some embodiments, the normal vector 1302 of a vertex 1202 can be created with one or several edges 1204 extending from that vertex 1202, and specifically by selecting a pair of edges 1204 extending from the vertex 1202 and using those edges 1204 to calculate a cross product. In some embodiments, a single normal vector can be generated for each vertex 1202 in the polygon mesh, and in some embodiments, a plurality of partial normal vectors can be generated for each vertex 1202 in the polygon mesh 1200. In some embodiments, and as depicted in FIG. 13, a vertex 1202 can have more than two edges 1204 extending from it, and thus, multiple partial normal vectors can be generated. In some embodiments, after a desired number of partial normal vectors have been generated for each vertex, these partial normal vectors can be combined to create the vertex normal vector 1302.

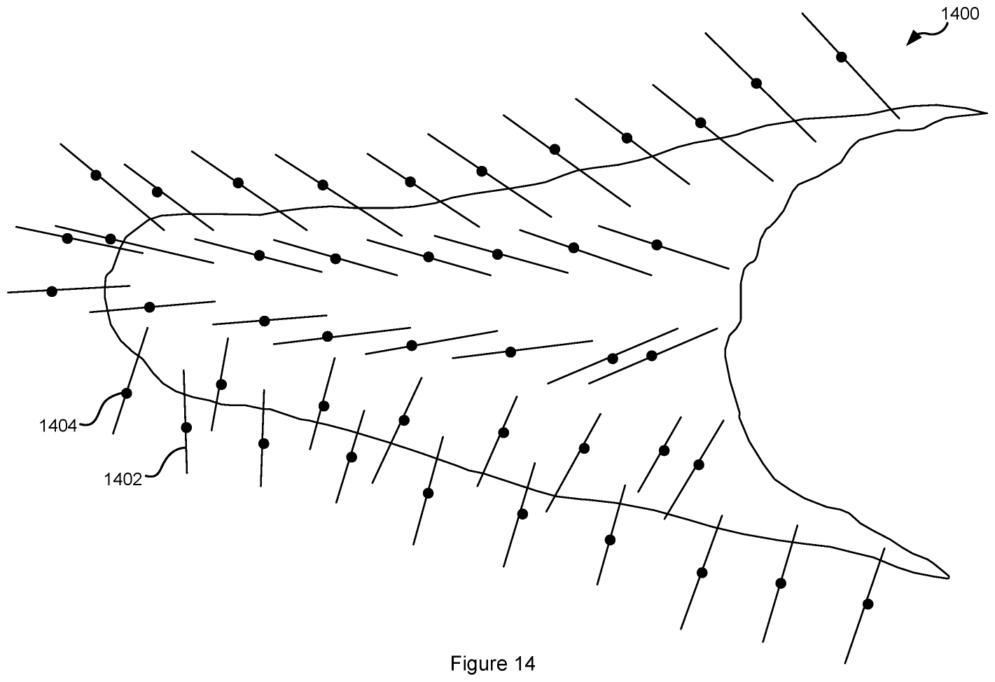

FIG. 14 illustrates one embodiment of a waypoint/delivery vector array 1400. After the vertex normal vectors 1302 have been created for the vertices 1202 in the polygon mesh 1200, a waypoint/delivery vector array 1400 is created. This includes the creation of delivery vectors 1402 and waypoints 1404. In some embodiments, the delivery vector 1402 indicates a direction for delivery of a spray treatment by the cryospray applicator 102. In some embodiments, during delivery of a treatment, the cryospray applicator 102 can be configured to deliver a spray treatment along the delivery vector 1402 of the cryospray applicator's 102 current location. In other words, the cryospray applicator 102 can be controlled such that when a cryospray treatment is delivered at a location the line of spray 304 is in the same direction as the delivery vector 1402 for the location.

In some embodiments, a waypoint 1404 can be a location in space that the cryospray applicator 102 should be moved to, or through during delivery of a cryospray treatment. In some embodiments, a waypoint can be at a location along a delivery vector 1402, and, in some embodiments, can be added at a location along the delivery vector 1402. The cryospray applicator 102 can move to or through one or several waypoints 1404 during delivery of a treatment. In some embodiments, the cryospray applicator 102 can dwell remain at a waypoint for some amount of time, which amount of time can be predetermined or based on information received from, for example, the one or several temperature detection features 212. Alternatively, in some embodiments, the cryospray applicator 102 can move through the waypoints 1404, and specifically, the cryospray applicator 102 can be continuously in motion as it travels through one or several waypoints 1404, however, the speed of this motion can vary based on, for example, information received from the one or several temperature detection features 212.

In some embodiments, a delivery vector can be created through the combination of a number of normal vectors 1302. Thus, in some embodiments, a delivery vector can be created by identifying a group of normal vectors, and combining the normal vectors in the group of normal vectors to form the delivery vector. A delivery vector can extend from its vertex. In some embodiments, the number of normal vectors 1302 in the group of normal vectors combined to create a delivery vector can vary based on one or several attributes of the spray treatment, and specifically can vary based on the treatment footprint. Specifically, in some embodiments vertex normal vectors 1302 can be combined in a number such that the size of the aggregated boxes 1006 of the combined vertex normal vectors 1302 is equal to, or approximately equal to the treatment footprint. In such embodiments, the size of the treatment footprint can be known, and based on the known size of the treatment footprint, the number of vertex normal vectors 1302 to be combined to create a single delivery vector 1402 can be determined. Alternatively, the number of normal vectors 1302 to be combined to create a single delivery vector 1402 can be preprogrammed and/or set by a user.

In some embodiments, the creation of waypoints includes the placing of a waypoint along each of the delivery vectors. This can include the identification of a position along a delivery vector that is a desired distance from the vertex, from the polygon mesh, and/or from the surface of the skin. In some embodiments, this desired distance can be held constant, and in some embodiments, this desired distance can vary. In some embodiments, the delivery vectors 1402 and waypoints 1404 can be stored.

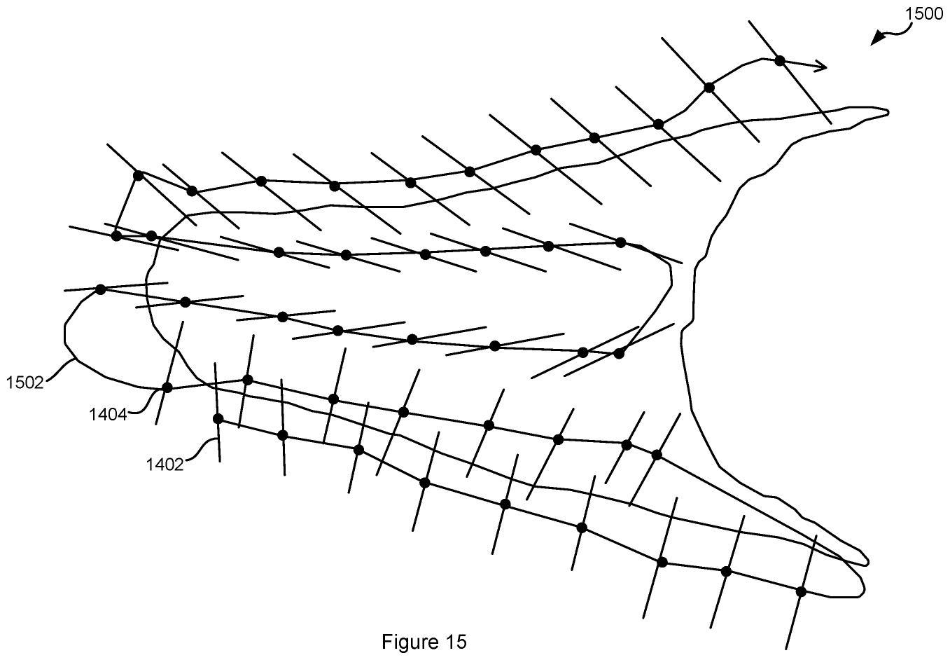

After the creation of the waypoints 1404 and the delivery vectors 1402, treatment paths 1502 can be created as indicated FIG. 15. In some embodiments, a treatment path can be created by the linking of a plurality of waypoints 1404. These waypoints 1404 can be linked in any desired manner, including, in some embodiments, in a sequential manner, according to the columns 1002 and the rows 1004 of the grid associated with the waypoints, according to adjacency, or the like. In some embodiments, the treatment path can include path portions intermediate between waypoints 1404 which control movement of the cryospray applicator 102 between the waypoints 1404. In some embodiments, the treatment paths can be created by the systematic advance between adjacent waypoints according to a pattern of the grid. This can be, for example, from left-to-right, right-to-left, top-to-bottom, or bottom to top. In some embodiments, the treatment path can wind through the grid until all waypoints have been linked.

In some embodiments, waypoints can be linked according to an evaluation one or several potential treatment paths 1502. In some embodiments, this can include the creation of a plurality of potential treatment paths 1502, and identifying and selecting the one of the plurality of potential treatment paths 1502 that is a best treatment path 1502 and/or that is an optimal treatment path 1502. In some embodiments, this best treatment path 1502 can be the treatment path 1502 that requires the least movement of the cryospray applicator 102 to align the axis 304 with the delivery vectors 1402 of the waypoints 1404 in the treatment path 1502, or in other words, that has the smallest aggregate difference in line of spray between adjacent delivery vectors 1402 in the treatment path 1502. In some embodiment, adjacent delivery vectors can include, for example, direct neighbor delivery vectors, and/or delivery vectors within a predetermined distance of each other.

In some embodiments, the creation of the one or several treatment paths 1502 can further the identification of one or several boundaries of the treatment area and/or one or several no-go zones. In some embodiments, the one or several treatment paths 1502 can be created, and specifically, the waypoints 1404 can be linked such that the treatment paths remain within the boundaries of the treatment area and/or stay out of the one or several no-go zones.

After the one or several treatment paths 1502 have been created, the controller 106 can control the mechanical arm 104 to move the cryospray applicator 102 according to the treatment path 1502. Specifically, the controller 106 can control the mechanical arm 104 to move the cryospray applicator 102 along the treatment path 1502 and through the waypoints 1404. The controller 106 can further control the mechanical arm 104 to move the cryospray applicator 102 such that when the cryospray applicator 102 delivers a treatment from a waypoint 1404, the line of spray 304 aligns with the delivery vector 1402 of that waypoint 1404.

In embodiments in which the cryospray applicator 102 includes one or several alignment sensors 208, one or several Time-of-flight (“TOF”) cameras 209, one or several distance sensors 210, and/or one or several temperature detection features 212, the movement of the cryospray applicator 102 can be controlled according to the treatment path and signals received from some or all of these cameras and/or sensors 208, 209, 210, 212. This can include, for example, determining with the one or several distance sensors 210 that cryospray applicator 102 is too close to, or too far from the treatment area. Alternatively, in embodiments in which the cryospray applicator 102 does not include one or several distance sensors 210, the TOF camera 209 can generate information during treatment, which information can be used to determine the distance between the cryospray applicator 102 and the treatment area. In such embodiments, information from the TOF camera 209 can be used to determine if the cryospray applicator 102 is moving through the waypoints 1404 and is aligning with the delivery vectors 1402. If the cryospray is not moving through the waypoints 1404 and/or is not aligning with the delivery vectors 1402, then the controller 106 can control the mechanical arm 104 to correct the movements of the cryospray applicator 102 to move through and/or to the waypoints 1404 and aligning with the delivery vectors 1402.

This description should not be interpreted as implying any particular order or arrangement among or between various steps or elements except when the order of individual steps or arrangement of elements is explicitly described. Different arrangements of the components depicted in the drawings or described above, as well as components and steps not shown or described are possible. Similarly, some features and sub-combinations are useful and may be employed without reference to other features and sub-combinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications may be made without departing from the scope of the claims below.

What is claimed is:

1. A method of controlling a skin cooling treatment system comprising a mechanical arm having a cryospray applicator coupled to a distal end of the mechanical arm, the method comprising:
receiving a point cloud generated from a portion of skin of a patient for receiving a skin cooling treatment;
generating a polygon mesh surface representative of the portion of skin of the patient from the point cloud, the polygon mesh surface comprising a plurality of linked vertices;
generating waypoints and delivery vectors based on the polygon mesh surface;
linking the waypoints to form a treatment path; and
delivering a skin treatment to the portion of skin according to the treatment path.

2. The method of claim 1, wherein the point cloud comprises a plurality of point clouds, each of the plurality of point clouds associated with a frame generated by a Time-of-flight camera.

3. The method of claim 2, further comprising organizing points from the point cloud into a grid defining a plurality of equally sized blocks.

4. The method of claim 3, wherein the points of the point cloud are unequally distributed among the plurality of equally sized blocks defined by the grid.

5. The method of claim 4, further comprising, for each block in the grid with at least one point, resolving the at least one point in the block to a vertex.

6. The method of claim 5, wherein the vertices have non-uniform depths.

7. The method of claim 5, wherein generating the polygon mesh comprises: identifying adjacent vertices; and linking adjacent vertices with edges.

8. The method of claim 7, wherein the polygon mesh surface comprises a triangle mesh.

9. The method of claim 8, further comprising generating a normal vector for at least some of the plurality of linked vertices of the polygon mesh surface.

10. The method of claim 9, wherein generating the normal vector for at least some of the plurality of linked vertices of the polygon mesh surface comprises: generating a plurality of partial normal vectors for each of the at least some of the plurality of linked vertices;
and for each of the at least some of the plurality of linked vertices combining the plurality of partial normal vectors to generate the normal vector for that linked vertex.

11. The method of claim 9, wherein the normal vector is created by selecting a pair of edges and calculating a cross product of that pair of edges.

12. The method of claim 11, wherein generating the delivery vectors comprises: identifying groups of normal vectors; and combining the normal vectors in each group of normal vectors to form a delivery vector.

13. The method of claim 12, wherein the groups of normal vectors comprise a number of normal vectors, and wherein the number of normal vectors corresponds to a treatment footprint of the cryospray applicator.

14. The method of claim 13, wherein generating the waypoints comprises placing a waypoint along each of the delivery vectors.

15. The method of claim 14, wherein placing a waypoint along each of the delivery vectors comprises, for each of the delivery vectors: identifying a position along the delivery vector a desired distance from a vertex of the delivery vector.

16. The method of claim 15, wherein all of the waypoints are positioned along their delivery vector at an equal distance from their vertex.

17. The method of claim 16, wherein linking the waypoints to form the treatment path comprises linking adjacent waypoints.

18. The method of claim 16, wherein linking the waypoints to form a treatment path comprises: generating a plurality of potential treatment paths; and determining an optimal treatment path from the plurality of potential treatment paths.

19. The method of claim 18, wherein determining the optimal treatment path comprises determining the one of the plurality of treatment path having the least movement of the cryospray applicator to a line of spray of the cryospray applicator with the delivery vectors in the treatment path.

20. The method of claim 19, wherein determining the optimal treatment path comprises identifying the one of the plurality of potential treatment paths having a smallest aggregate difference between adjacent delivery vectors.

21. The method of claim 20, wherein forming the treatment path comprises identifying at least one no-go zone; and linking waypoints to avoid the at least one no-go zone.

22. A skin cooling treatment system comprising:
a mechanical arm having a proximal end and a distal end;
a cryospray applicator coupled to the distal end of the mechanical arm, the cryospray applicator comprising an array of orifices, the cryospray applicator movable by the mechanical arm to deliver a spray of cryogen to a portion of an area of skin tissue for treatment; and
a processor configured to:
receive a point cloud generated from a portion of skin of a patient for receiving a skin cooling treatment;
generate a polygon mesh surface representative of the portion of skin of the patient from the point cloud, the polygon mesh surface comprising a plurality of linked vertices;
generate waypoints and delivery vectors based on the polygon mesh surface;
link the waypoints to form a treatment path; and
deliver a skin treatment to the portion of skin according to the treatment path.

23. The system of claim 22, wherein the point cloud comprises a plurality of point clouds, each of the plurality of point clouds associated with a frame generated by a Time-of-flight camera.

24. The system of claim 23, wherein the processor is further configured to organize points from the point cloud into a grid defining a plurality of equally sized blocks.

25. The system of claim 24, wherein the points of the point cloud are unequally distributed among the equally sized block defined by the grid.

26. The system of claim 25, wherein the processor is further configured to, for each block in the grid with at least one point, resolve the at least one point in the block to a vertex.

27. The system of claim 26, wherein the vertices have non-uniform depths.

28. The system of claim 26, wherein generating the polygon mesh comprises: identifying adjacent vertices; and linking adjacent vertices with edges.

29. The system of claim 28, wherein the polygon mesh surface comprises a triangle mesh.

30. The system of claim 29, wherein the processor is further configured to generate a normal vector for at least some of the plurality of linked vertices of the polygon mesh surface.

31. The system of claim 30, wherein generating the normal vector for at least some of the plurality of linked vertices of the polygon mesh surface comprises: generating a plurality of partial normal vectors for each of the at least some of the plurality of linked vertices; and for each of the at least some of the plurality of linked vertices combining the plurality of partial normal vectors to generate the normal vector for that linked vertex.

32. The system of claim 30, wherein the normal vector is created by selecting a pair of edges and calculating a cross product of that pair of edges.

33. The system of claim 32, wherein generating the delivery vectors comprises: identifying groups of normal vectors; and combining the normal vectors in each group of normal vectors to form a delivery vector.

34. The system of claim 33, wherein the groups of normal vectors comprise a number of normal vectors, and wherein the number of normal vectors corresponds to a treatment footprint of the cryospray applicator.

35. The system of claim 34, wherein generating the waypoints comprises placing a waypoint along each of the delivery vectors.

36. The system of claim 35, wherein placing a waypoint along each of the delivery vectors comprises, for each of the delivery vectors: identifying a position along the delivery vector a desired distance from a vertex of the delivery vector.

37. The system of claim 36, wherein all of the waypoints are positioned along their delivery vector at an equal distance from their vertex.

38. The system of claim 37, wherein linking the waypoints to form the treatment path comprises linking adjacent waypoints.

39. The system of claim 37, wherein linking the waypoints to form a treatment path comprises: generating a plurality of potential treatment paths; and determining an optimal treatment path from the plurality of potential treatment paths.

40. The system of claim 39, wherein determining the optimal treatment path comprises determining the one of the plurality of treatment path having the least movement of the cryospray applicator to a line of spray of the cryospray applicator with the delivery vectors in the treatment path.

41. The system of claim 40, wherein determining the optimal treatment path comprises identifying the one of the plurality of potential treatment paths having a smallest aggregate difference between adjacent delivery vectors.

42. The system of claim 41, wherein forming the treatment path comprises identifying at least one no-go zone; and linking waypoints to avoid the at least one no-go zone.
